(19) United States
(12) Patent Application Publication
(10) Pub. No.: US 2017/0151136 A1
(43) Pub. Date: Jun. 1, 2017

(54) SILICA-CONTAINING COMPOSITE NANOPARTICLES, AND HYDROGEL MOISTURIZING PATCH CONTAINING SAME

(71) Applicant: Joo Won KIM, Gyeonggi-do (KR)

(72) Inventor: Joo Won KIM, Gyeonggi-do (KR)

(21) Appl. No.: 15/310,599

(22) PCT Filed: May 12, 2015
(86) PCT No.: PCT/KR2015/004732

§ 371 Date: Feb. 3, 2017

(30) Foreign Application Priority Data
May 14, 2014 (KR) 10-2014-0058013

(51) Int. Cl. A61K 8/25; A61Q 19/00; A61K 8/81; A61K 8/02; A61K 8/04

(52) U.S. Cl. CPC A61K 8/25; A61K 8/0241; A61K 8/0208; A61K 8/042; A61K 8/8152; A61Q 19/00; A61K 2800/624; A61K 2800/651

(57) ABSTRACT

The present invention relates to silica/zwitterionic polymer composite nanoparticles, a preparation method therefor, and a hydrogel moisturizing patch containing the same. The silica/zwitterionic polymer composite nanoparticles of the present invention reduces the evaporation velocity of water by displaying a strong binding force to water and exhibits water retention and skin barrier function reinforcement effects, and thus can be used as an artificial moisturizing factor for preparing a hydrogel moisturizing patch or various cosmetic formulations pursuing similar skin effects thereto.

### TECHNICAL FIELD

The present invention relates to silica-containing composite nanoparticles, and a hydrogel moisturizing patch containing the same.

### BACKGROUND ART

Skin not only protects our body from external harmful environment, but also prevents moisture flow out of our body. The reason that the skin composed mostly of organic structures exerts this excellent protection and blocking abilities is because stratum corneum exists in an outermost layer of the skin. The stratum corneum is a layered structure in which keratinocytes are fixed by a lipid layer and has a thickness of 15 to 30 μm.

A hydrogel patch applied on the skin is able to replace a unique skin protection performance of the stratum corneum. In particular, an outer skin layer allows to be saturated with moisture, which facilitates smooth skin regeneration activity inside the skin. To further enhance the performance of the patch, an ability to retain moisture needs to be excellent like the stratum corneum. The keratinocytes constituting the stratum corneum have a disk-like flat shape and are stacked one upon another to form a multilayer structure. An inside of the keratinocytes consists of keratin, a natural moisturizing factor, protein, etc., wherein the keratin acts as a structural reinforcement, and the natural moisturizing factor exhibits strong hygroscopicity to saturate the inside of the keratinocytes with moisture. Therefore, the inside of keratinocytes is a hydrogel saturated by moisture. An excellent moisture retention ability of the keratinocyte is because the natural moisturizing factor is contained on the hydrogel, and the stratum corneum in which the keratinocytes form a large area layered structure may refer to a huge moisture patch. The natural moisturizing factor is included with 20-30% in the keratinocytes and has strong hydrogen bond and ionic bond with moisture, thereby blocking moisture evaporation from the keratinocytes. Therefore, if a hydrogel system containing an artificial moisturizing factor capable of replacing a role of the natural moisturizing factor is developed, it is expected to develop a technology of a highly functional moisture patch.

Various types of moisture patches have been developed to date. The hydrogel patch produced based on a water-soluble polymer imparts a feeling of moisturization by hydrating the skin outer layer immediately upon application to the skin. However, if there is no additional improvement on the system, it is difficult to retain a hydration state for a long period of time. In order to solve the problem, a patch containing a fat-soluble patch or an emulsion has been developed. In order to retain moisture for a long period of time, it is required to provide a strong binding force between water molecules and matrix.

Therefore, there is a constant demand for development of the artificial moisturizing factor capable of blocking moisture evaporation in the hydrogel system.

### DISCLOSURE

### Technical Problem

An object of the present invention is to provide an artificial moisturizing factor capable of blocking moisture evaporation in a hydrogel system, and a production method thereof.

Another object of the present invention is to provide a hydrogel moisturizing patch including the artificial moisturizing factor.

### Technical Solution

In one general aspect, there is provided a silica/zwitterionic polymer composite nanoparticle including: a silica; and a zwitterionic polymer thin-film-coated by crosslinking on a surface of the silica particle.

In another general aspect, there is provided a production method of a silica/zwitterionic polymer composite nanoparticle including: 1) coupling silica with a silane compound to introduce an amine group onto a surface of the silica particle; 2) performing a condensation reaction of the silica particle obtained in step 1) with trichloroacetyl isocyanate to introduce a trichloroacetyl group onto the surface of the silica particle; and 3) polymerizing the silica particle obtained in step 2) with a monomer containing a cation and an anion in the same molecule in the presence of a crosslinking agent to introduce a zwitterionic polymer thin film layer onto the surface of the silica particle.

In another general aspect, there is provided a hydrogel moisturizing patch including the silica/zwitterionic polymer composite nanoparticle as described above.

### Advantageous Effects

The silica/zwitterionic polymer composite nanoparticles of the present invention exhibit a strong binding force with moisture to reduce an evaporation velocity of moisture, and show moisture retention and skin barrier function reinforcement effects, and thus, may be effectively used as an artificial moisturizing factor for producing a hydrogel moisturizing patch or various cosmetic compositions pursuing similar skin effects thereto.

Further, the silica/zwitterionic polymer composite nanoparticle according to the present invention includes silica, and a zwitterionic polymer thin-film-coated by crosslinking on a surface of the silica particle to exhibit low viscosity behavior and elastic behavior, thereby having improved fluid fluidity while reducing stickiness which is a unique feeling when using a polymer.

### DESCRIPTION OF DRAWINGS

FIG. 1 is a diagram of a hydrogel patch system design using silica/zwitterionic polymer composite nanoparticles as an artificial moisturizing factor (AMF).

FIG. 2(a) is a transmission electron microscope image (scale bar: 50 nm) of pure silica nanoparticles, FIGS. 2(b) and 2(c) are transmission electron microscope images (scale bar: 50 nm and 200 nm) of the silica/zwitterionic polymer composite nanoparticles produced in Example 1.

FIG. 3 shows result of thermogravimetric analysis of the silica/zwitterionic polymer composite nanoparticles depending on content of a silica/zwitterionic polymer.

FIG. 4 is a graph showing a change in interfacial tension of the silica/zwitterionic polymer composite nanoparticles produced in Examples 1 to 3.

FIG. 5 is a graph showing an evaporation velocity of moisture of the silica/zwitterionic polymer composite nanoparticles produced in Examples 2 and 3.

FIG. 6 shows viscosity behavior relative to shear stress of the silica/zwitterionic polymer composite nanoparticles produced in Example 2 and Comparative Example 1.

FIG. 7 shows storage modulus relative to oscillation strain of the silica/zwitterionic polymer composite nanoparticles produced in Example 2 and Comparative Example 1.

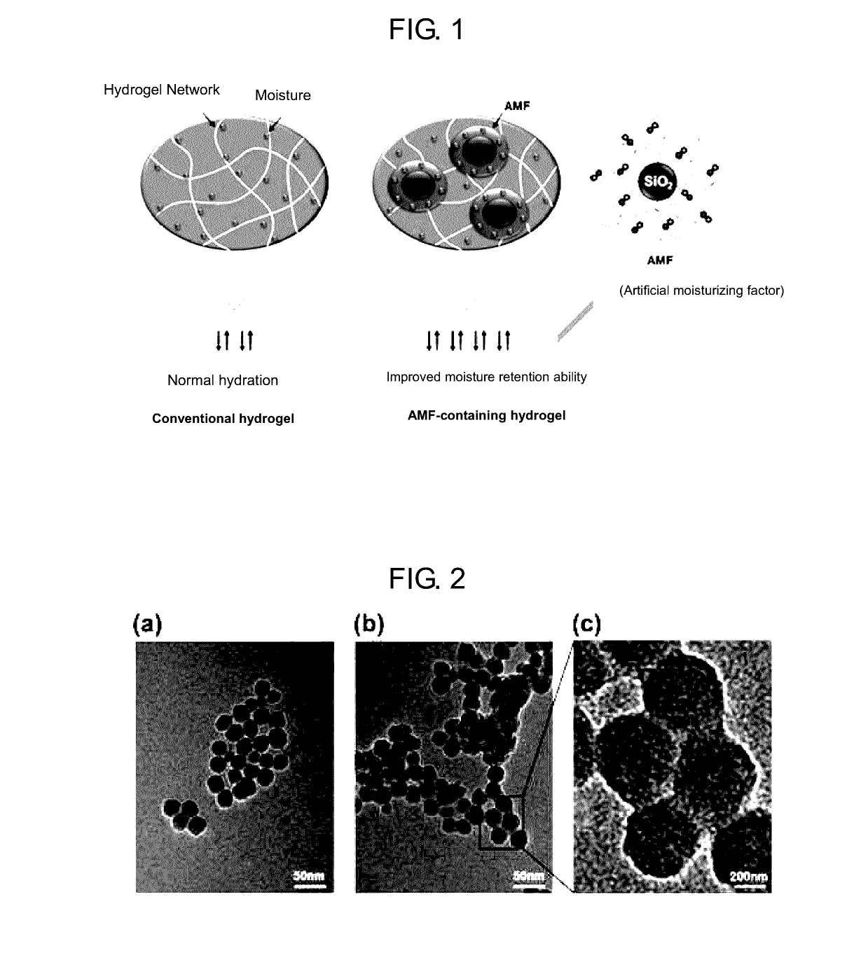

### DETAILED DESCRIPTION OF THE INVENTION

The present invention provides a silica/zwitterionic polymer composite nanoparticle including: a silica; and a zwitterionic polymer thin-film-coated by crosslinking on a surface of the silica, using a monomer containing a cation and an anion in the same molecule.

The silica/zwitterionic polymer composite nanoparticle may include the silica and the zwitterionic polymer at a ratio of 1:0.5 to 1:5 (w/w), preferably, 1:0.5 to 1:3 (w/w), and more preferably, 1:0.5 to 1:1 (w/w).

The silica/zwitterionic polymer composite nanoparticle according to the present invention may have a diameter of 20 to 30 nm, and preferably, about 20 nm, in a dry state and have a diameter of 50 nm to 100 nm after being hydrated in water. A swelling rate may be easily controlled to a desired level as polymer chains are bonded to each other by the crosslinking of the thin-film-coated zwitterionic polymer, such that water may be effectively trapped in a crosslinking chain, thereby exhibiting more improved moisturizing effect.

The silica usable in the present invention is not limited as long as it has an average particle diameter of 20 to 25 nm (for example, 22 nm), but it is preferred that the silica has a negative surface potential, and it is possible to use a negative potential silica dispersed in a water phase.

Term “zwitterionic polymer” used herein means a polymer synthesized by a single polymerization or copolymerization process of a monomer containing a cation and an anion in the same molecule.

The zwitterionic polymer according to an exemplary embodiment of the present invention is not limited as long as it is a polymer polymerized from a monomer containing a cation and an anion in the same molecule. As preferred examples, the zwitterionic polymer may be 2-methacryloyloxyethyl phosphorylcholine (MPC), 2-methacryloyloxyethyl phosphatidylcholine, 2-(methacryloyloxy)ethyl-2′-(trimethylammonio)ethylphosphate, 2-methacryloyloxyethyl phosphoethanolamine, etc. More preferably, the zwitterionic polymer may be 2-methacryloyloxyethyl phosphorylcholine (MPC) since it is able to provide excellent moisturizing effect and to be thin-film-coated by crosslinking to be capable of maximizing inhibition of moisture evaporation in a hydrogel system.

The zwitterionic polymer according to an exemplary embodiment of the present invention may be crosslinked to silica and thin-film-coated (FIG. 2), wherein the zwitterionic polymer has a hydrogel form. According to an exemplary embodiment of the present invention, as results of thermogravimetric analysis of composite nanoparticles according to the present invention, a thin film including the zwitterionic polymer may be coated on silica surface with 12 to 20 wt %, preferably, 15 to 18 wt % on the basis of the total weight.

Meanwhile, the present invention provides a production method of a silica/zwitterionic polymer composite nanoparticle including: 1) coupling silica with a silane compound to introduce an amine group onto a surface of the silica particle; 2) performing a condensation reaction of the silica particle obtained in step 1) with trichloroacetyl isocyanate to introduce a trichloroacetyl group onto the surface of the silica particle; and 3) polymerizing the silica particle obtained in step 2) with a monomer containing a cation and an anion in the same molecule in the presence of a crosslinking agent to introduce a zwitterionic polymer thin film layer onto the surface of the silica particle.

In the method, in step 1), a silica-silane coupling reaction may be performed to increase reactivity, such that an amine group may be introduced onto the surface of the silica particle.

The silica is converted into a powder form by evaporating moisture, and then is dispersed in an organic solvent by irradiating ultrasonic wave, etc., thereby preparing a silica dispersion having 3 to 5 wt %, for example, about 3 wt %. Here, the organic solvent may be one or more organic solvents selected from toluene, chloroform, methylene chloride, tetrahydrofuran, xylene, etc., but is not limited thereto. Toluene is preferred in view of solubility and dispersibility of a reaction material.

Then, 1 to 300 parts by weight of the silane compound may be added on the basis of 100 parts by weight of the dispersed silica, followed by the silane coupling reaction at 110 to 120° C. for 8 to 9 hours, thereby introducing the amine group onto the surface of the silica particle. The silane compound is not limited as long as it is capable of introducing the amine group onto the surface of silica particle. However, the silane compound may be one or more selected from 3-aminopropyltriethoxysilane (APS), 3-aminopropyltrimethoxysilane, 4-aminobutyltriethoxysilane, etc., and preferably, APS in view of reactivity and reaction yield.

Step 2) is a step of introducing a polymerization initiation site onto the surface of the silica particle, and a trichloroacetyl group may be introduced onto the surface of silica particle by performing a condensation reaction of the amine group of the silica particle obtained in step 1) with a trichloroacetyl isocyanate group.

The silica particles obtained in step 1) are dispersed in the organic solvent by irradiating ultrasonic wave, etc., in the same manner as described above, thereby preparing a silica dispersion having 3 to 5 wt %, for example, about 3 wt %. Here, the organic solvent may be one or more organic solvents selected from toluene, chloroform, methylene chloride, tetrahydrofuran, xylene, etc.

Then, 10 to 200 parts by weight of trichloroacetyl isocyanate on the basis of 100 parts by weight of the dispersed silica may be subjected to the condensation reaction, in the presence of dibutyltin dilaurate at 80 to 90° C. for 8 to 9 hours, such that the trichloroacetyl group may be introduced onto the surface of the silica particle as the polymerization initiation site.

Here, the dibutyltin dilaurate may be used as a urethane reaction catalyst.

In step 3), the silica obtained in step 2) may be subjected to a polymerization reaction by adding the monomer containing a cation and an anion in the same molecule and a crosslinking agent to introduce a zwitterionic polymer thin film layer onto the surface of the silica particle.

The silica particles obtained in step 2) are dispersed in a solvent to prepare a dispersion having 6 to 8 wt %, for example, about 6 wt %, and then the monomer containing a cation and an anion in the same molecule and the crosslinking agent are added thereto. The solvent is not limited as long as it has a high degree of dispersion for the silica particle and a high solubility for the monomer and the crosslinking agent. As a non-limiting example, the solvent may be C1-C3 lower alcohol, and preferably, one or more solvents selected from methanol, ethanol, and isopropanol.

In the production method of the silica/zwitterionic polymer composite nanoparticle according to an exemplary embodiment of the present invention, it is the most preferred that the monomer containing a cation and an anion in the same molecule is MPC and the crosslinking agent is divinylbenzene (DVB).

The monomer and the crosslinking agent may be added in a content of 1 to 500 parts by weight on the basis of 100 parts by weight of the silica, and may be polymerized with the silica particle at 65 to 70° C. for 12 to 13 hours. Here, the crosslinking agent may be used in a content of 0.1 to 50 wt % on the basis of the total weight of the monomer and the crosslinking agent. In order to improve physical properties of the crosslinked thin film to have improved moisturizing ability and moisture retention ability, the crosslinking agent may preferably have a content of 0.1 to 30 wt %, and more preferably, 1 to 15 wt %. If the content is out of the above-described range, hydrophobicity of the thin film layer may be increased, and rather, the moisture may be easily evaporated. Therefore, the crosslinking agent preferably has the content within the above-described range.

In step 3), a catalyst may be further used to improve a reaction velocity. The catalyst is not limited as long as it is capable of improving the reaction rate, but may be preferably Mo(CO)₆. The catalyst may have a content of 0.01 to 5 wt % on the basis of the total weight, preferably 0.01 to 3 wt %, and more preferably 0.01 to 1 wt %, but the present invention is not limited thereto.

The silica/zwitterionic polymer composite nanoparticle according to the present invention exhibits a strong binding force with moisture to reduce an evaporation velocity of moisture, and shows moisture retention and skin barrier function reinforcement effects, which is effectively usable as an artificial moisturizing factor for producing a hydrogel moisturizing patch or various cosmetic formulations pursuing similar skin effects thereto.

Therefore, the present invention provides a moisturizing cosmetic composition including the silica/zwitterionic polymer composite nanoparticle. The moisturizing cosmetic composition may be, for example, a hydrogel moisturizing patch, a water patch and a water wrap, etc., and preferably, a hydrogel moisturizing patch since it is capable of showing more improved moisturizing effect.

In the case of a fluid containing the silica/zwitterionic polymer composite nanoparticle according to the present invention, it is possible to form a dense hydration layer on the silica surface by the crosslinked zwitterionic polymer thin film, such that the strong binding force with moisture may be exhibited to reduce the evaporation velocity of moisture, thereby showing more improved moisturizing effect.

Further, due to the crosslinked zwitterionic polymer thin film, it is possible to exhibit improved fluid fluidity while reducing stickiness which is a unique feeling when using the polymer.

Hereinafter, the present invention will be described in more detail by the following Examples. However, the following Examples are merely described for illustrative purposes, and the scope of the present invention is not limited thereto.

### EXAMPLE 1

### Synthesis of Artificial Moisturizing Factor (Silica/Zwitterionic Polymer Composite Nanoparticle)

Reagent

Silica (Ludox CL-X) was available from Aldrich. The silica had an average particle diameter of 22 nm, and the silica had a negative surface potential, and sodium was used as a stabilizing counter ion. The silica had a form in which it was dispersed in a water phase, and had a concentration of 45 wt %.

All of 2-methacryloyloxyethyl phosphorylcholine (MPC), 3-aminopropyltriethoxysilane (APS), trichloroacetyl isocyanate, dibutyltin dilaurate, and divinylbenzene (DVB) were available from Sigma-Aldrich, and the dibutyltin dilaurate was used as a urethane reaction catalyst, and the DVB was used as a crosslinking agent.

Step 1) Introduction of Amine Group onto Surface of Silica Particle

An amine group was introduced by a silica-silane coupling reaction in order to increase reactivity of the surface of the silica particle.

First, moisture contained in the silica particle (Ludox CL-X) was evaporated under reduced pressure at room temperature to obtain a powder form of silica. The thus-obtained silica powder was put into toluene and re-dispersed by irradiating ultrasonic wave at 35° C. for 10 minutes using a probe type ultrasonic wave irradiator (Sonics & Material Inc., VCX500, USA).

A concentration of the silica particles was fixed to 3 wt %. Next, in order to introduce the amine group, 150% of APS relative to a weight of the silica particle was added and reacted on the surface of the silica particle at 110° C. for 8 hours. The silica particle onto which the amine group was introduced obtained through the above process was washed and recovered through repetitive centrifugation at 5,000 rpm by 5 or more times.

Step 2) Introduction of Trichloroacetyl Group onto Surface of Silica Particle

In order to introduce a polymerization initiation site onto the silica particle, the silica particle obtained in step 1) was subjected to a condensation reaction with trichloroacetyl isocyanate.

First, the silica particles onto which the amine group was introduced obtained in step 1) were put into toluene and re-dispersed by irradiating ultrasonic wave at 35° C. for 10 minutes using a probe type ultrasonic wave irradiator (Sonics & Material Inc., VCX500, USA). A concentration of the silica particles was fixed to 3 wt %. Then, 80% of trichloroacetyl isocyanate relative to a weight of the re-dispersed silica particles was added together with 1 wt % of dibutyltin dilaurate on the basis of the total weight of the toluene including the silica particles, and the obtained mixture was stirred at 80° C. for 8 hours to perform a condensation reaction. The silica particle onto which the trichloroacetyl group was introduced obtained through the above process was washed and recovered through repetitive centrifugation at 5,000 rpm by 5 or more times.

Step 3) Introduction of Polymer Thin Film Layer by Surface Polymerization

The silica particles onto which the trichloroacetyl group was introduced obtained in step 2) were re-dispersed in ethanol. Here, a concentration of the silica particles was fixed to 6 wt %. Then, MPC and DVB were added to the silica dispersion after the re-dispersion. Here, a concentration of DVB was 10 wt % relative to MPC, and 0.05 wt % of Mo(CO)₆ was added as a catalyst on the basis of the total weight. Finally, a content ratio of silica and MPC was adjusted to 1/0.5 (w/w). Oxygen was removed by injecting argon into the reactor, and then, a polymerization reaction for polymer was performed at 70° C. for 12 hours, thereby finally synthesizing silica/zwitterionic polymer composite nanoparticle in which the zwitterionic polymer was coated on the silica surface, wherein the zwitterionic polymer had a chemical name of poly(2-methacryloyloxy)ethyl-2-(trimethylammonio)ethylphosphate-co-divinylbenzene.

### EXAMPLE 2

### Synthesis of Artificial Moisturizing Factor (Silica/Zwitterionic Polymer Composite Nanoparticle)

A silica/zwitterionic polymer composite nanoparticle in which a zwitterionic polymer was coated onto a silica surface was synthesized in the same manner as Example 1 except that the content ratio of silica and MPC was adjusted to 1/1 (w/w) in step 3 of Example 1.

### EXAMPLE 3

### Synthesis of Artificial Moisturizing Factor (Silica/Zwitterionic Polymer Composite Nanoparticle)

A silica/zwitterionic polymer composite nanoparticle in which a zwitterionic polymer was coated onto a silica surface was synthesized in the same manner as Example 1 except that the content ratio of silica and MPC was adjusted to ½ (w/w) in step 3 of Example 1.

### EXAMPLE 4

### Synthesis of Artificial Moisturizing Factor (Silica/Zwitterionic Polymer Composite Nanoparticle)

A silica/zwitterionic polymer composite nanoparticle in which a zwitterionic polymer was coated onto a silica surface was synthesized in the same manner as Example 1 except that the content ratio of silica and MPC was adjusted to ⅓ (w/w) in step 3 of Example 1.

The thus-produced silica/zwitterionic polymer composite nanoparticle was synthesized by a combination of living polymerization and seeded polymerization. The silica/zwitterionic polymer composite nanoparticle had a diameter of 20 nm in the dry state and 50 to 100 nm after being hydrated in water. The zwitterionic polymer was coated on the silica surface while having a hydrogel form, in a thickness of several nanometers (for example, 0.5 to 5 nm).

A transmission electron microscope (TEM) image of the pure silica nanoparticle (a) and TEM images of the silica/zwitterionic polymer composite nanoparticles (b and c) produced in Example 1 were shown in FIG. 2. As shown in FIG. 2, it could be appreciated that the zwitterionic polymer was introduced onto the surface of the silica nanoparticle while having a thin film form.

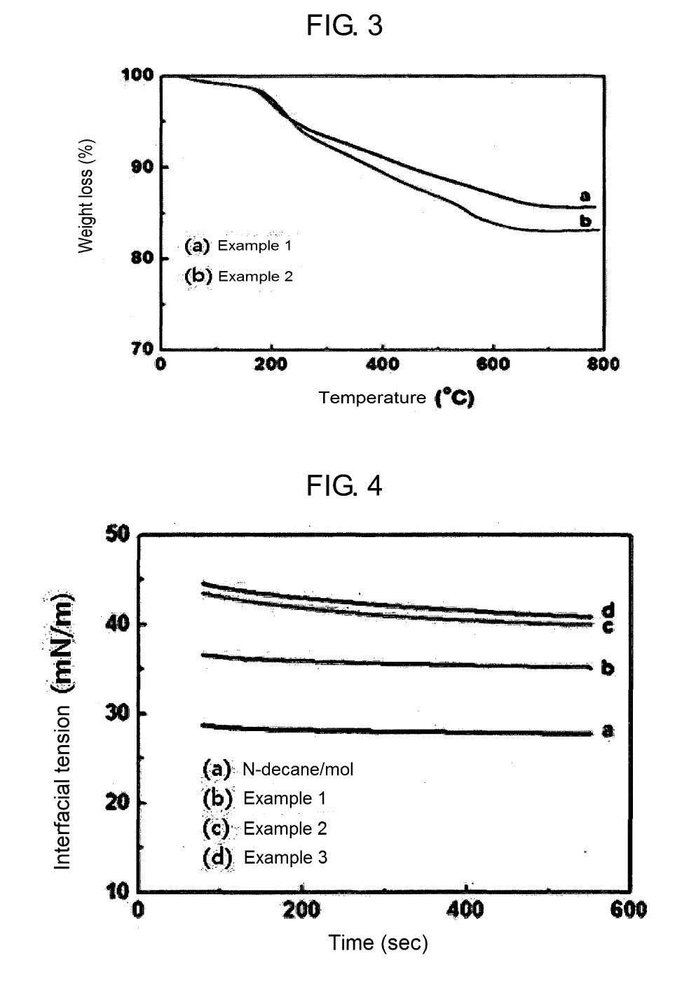

Further, the silica/zwitterionic polymer composite nanoparticles of Examples 1 and 2 wherein the ratios of silica/zwitterionic polymer were 1:0.5 and 1:1 (w/w), respectively, were subjected to thermogravimetric analysis (TGA) (Q500, TA instrument (USA)), and results were shown in FIG. 3.

As shown in FIG. 3, the zwitterionic polymer coated onto the silica surface burned as the temperature was higher, and thus, the weight was reduced, and it could be quantitatively confirmed that a zwitterionic polymer layer was introduced with about 15 to 18 wt %.

### COMPARATIVE EXAMPLE 1

### Synthesis of Non-Crosslinked Silica/Zwitterionic Polymer Composite Nanoparticle

A silica/zwitterionic polymer composite nanoparticle in which a zwitterionic polymer was linearly coated onto a silica surface was synthesized in the same manner as Example 2 except that the DVB was not used in step 3 of Example 2.

Further, a moisture retention performance of the silica/zwitterionic polymer composite nanoparticles (a, b, and c) produced in Examples 1 to 3 was evaluated, and properties of fluids including the nanoparticles were evaluated as follows.

Measurement of Interfacial Tension

In order for the silica/zwitterionic polymer composite nanoparticle to effective exhibit a moisture retention ability, it was required to have an excellent ability to hydrate water in a water phase. In order to hydrate water in the water phase, a physical bonding with water molecules was essentially required. If the physical bonding phenomenon was generated, an interfacial tension of water was increased.

Accordingly, the silica/zwitterionic polymer composite nanoparticles produced in Examples 1 to 3 were dispersed in water at a concentration of 0.1 wt %, respectively, and a change in oil-water interface tension was measured.

As a result, as shown in FIG. 4, it could be appreciated that the interfacial tension was increased as the ratio of the zwitterionic polymer was increased. The reason was because the thin film layer including the crosslinked zwitterionic polymer formed a strong bond with water molecules to increase condensation force between water molecule and water molecule, and thus, the silica/zwitterionic polymer composite nanoparticle was positioned on a bulk rather than positioned at an interface, thereby changing properties of water on the bulk.

2. Measurement of Evaporation Velocity of Moisture

Meanwhile, in order to confirm whether an increase in binding force between the water molecules affected an actual evaporation velocity of the water molecules, an evaporation velocity of moisture was measured.

In order to determine an exact evaporation velocity of moisture, moisture permeability was measured on pure water, the silica/zwitterionic polymer (1:1, w/w) of Example 2 and the silica/zwitterionic polymer (1:2, w/w) of Example 3 using a moisture permeability tester (Alt-lab). Here, the silica/zwitterionic polymers were dissolved in water at a concentration of 0.1%, respectively, and used for the test.

Each of the three test aqueous solutions was placed on an electronic scale, and a temperature (40° C.) and humidity (30%) were accurately set, and then, the system was sealed. The evaporation velocity of moisture was determined by measuring weight loss over time.

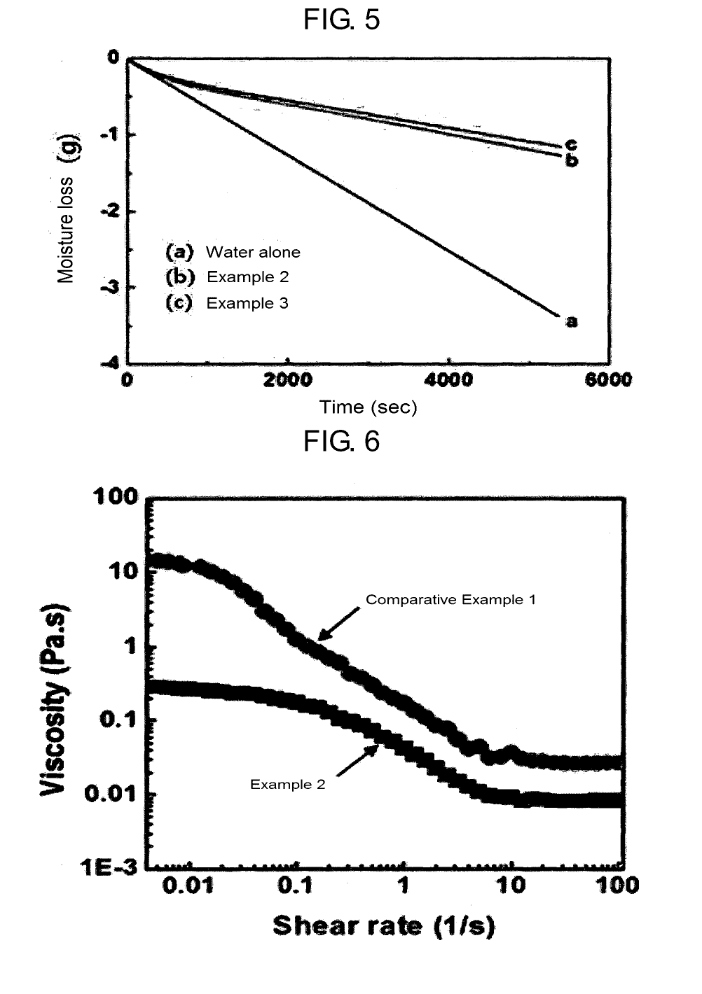

As a result, as shown in FIG. 5, in the case of pure water (a), a linear decrease in moisture occurred over time. It indicated that the hydrogen bonds between the water molecules were reduced according to the linear constant relationship, leading to moisture evaporation. On the contrary, in the case of the aqueous solutions (b and c) in which the silica/zwitterionic polymer nanoparticles were dispersed, the evaporation velocity of the aqueous solutions followed that of water at the beginning of the evaporation, and then, slowly reduced. The reduced evaporation velocity continued until all of the water had evaporated. It could be appreciated that the difference in the evaporation velocity had similar behavior to the change in surface tension.

3. Viscosity Behavior of Fluid

In order to confirm the viscosity behavior of the fluid containing the silica/zwitterionic polymer composite nanoparticle, first, the silica/zwitterionic polymer composite nanoparticles synthesized in Example 2 and Comparative Example 1 were dispersed in water at a concentration of 45 vol %, respectively. The uniform dispersion could be obtained by irradiating ultrasonic wave at strength of 500 W for 1 minute. A rheological behavior of the fluid containing the nanoparticle produced as described above was measured using a AR200/DHR-3 rheometer (TA Instrument, USA) in a stress-control mode. A cone plate to be used had a geometric angle of 1 degree. A solvent trap was installed to prevent moisture evaporation during the measurement. Then, the viscosity behavior relative to shear stress was observed while changing the shear stress from 0.001 to 100 s⁻¹. All experiments were performed at room temperature (23° C.).

As a result, as shown in FIG. 6, the fluid containing the silica/zwitterionic polymer composite nanoparticle according to Example 2 had the viscosity behavior about 20 times lower than that of the non-crosslinked silica/zwitterionic polymer composite nanoparticle according to Comparative Example 1.

4. Elastic Behavior of Fluid

The elastic behavior of the nanoparticle-containing fluid was observed in the same manner as the viscosity behavior of the fluid as described above except that oscillation strain was changed from 0.1 to 100%.

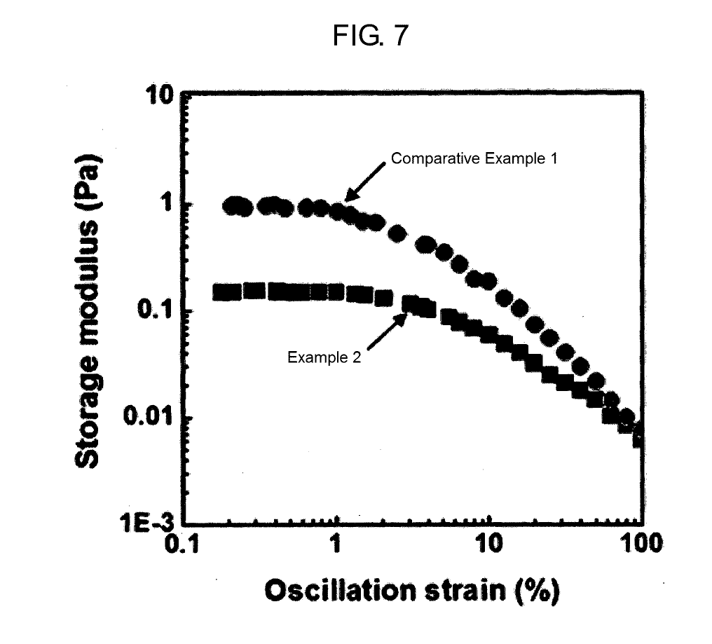

As a result, as shown in FIG. 7, the fluid containing the silica/zwitterionic polymer composite nanoparticle according to Example 2 had the elastic behavior about 10 times lower than that of the non-crosslinked silica/zwitterionic polymer composite nanoparticle according to Comparative Example 1.

These experimental results showed that the silica/zwitterionic polymer composite nanoparticle had a strong binding force with water in the water phase, and exhibited a function of reducing the evaporation velocity of water molecules, and further, showed improved fluid fluidity while reducing stickiness which was a unique feeling when using the polymer.

As the natural moisturizing factor acts a major role in expression of moisture retention ability on the hydrogel inside the keratinocytes, it could be confirmed that the silica/zwitterionic polymer composite nanoparticle developed in the present invention exhibited a strong binding force with moisture, and had a performance of reducing the evaporation velocity of moisture, which is useful as the artificial moisturizing factor.

Therefore, the cosmetic composition including the artificial moisturizing factor of the present invention is expected to simultaneously implement an effect of reinforcing moisture retention and an effect of reinforcing skin barrier function, and thus, may be effectively used as a raw material for a hydrogel patch and various cosmetic formulations for moisturizing.

1. A silica/zwitterionic polymer composite nanoparticle comprising:
a silica; and
a zwitterionic polymer thin-film-coated by crosslinking on a particle surface of the silica.

2. The silica/zwitterionic polymer composite nanoparticle of claim 1, wherein the composite nanoparticle includes the silica and the zwitterionic polymer at a ratio of 1:0.5 to 1:5 (w/w).

3. The silica/zwitterionic polymer composite nanoparticle of claim 1, wherein the zwitterionic polymer is thin-film-coated with 12 to 20 wt %.

4. The silica/zwitterionic polymer composite nanoparticle of claim 1, wherein the zwitterionic polymer is derived from 2-methacryloyloxyethyl phosphorylcholine.

5. The silica/zwitterionic polymer composite nanoparticle of claim 1, wherein the silica/zwitterionic polymer composite nanoparticle has an average diameter of 20 to 30 nm in a dry state and has an average diameter of 50 to 100 nm after being hydrated in water.

6. A production method of a silica/zwitterionic polymer composite nanoparticle comprising:
1) coupling silica with a silane compound to introduce an amine group onto a particle surface of the silica;
2) performing a condensation reaction of the silica particle obtained in step 1) with trichloroacetyl isocyanate to introduce a trichloroacetyl group onto the surface of the silica particle; and
3) polymerizing the silica particle obtained in step 2) with a monomer containing a cation and an anion in the same molecule in the presence of a crosslinking agent to introduce a zwitterionic polymer thin film layer onto the surface of the silica particle.

7. The production method of claim 6, wherein step 1) is performed by adding the silane compound to the silica, the silane compound having a content of 1 to 300 parts by weight on the basis of 100 parts by weight of the silica, followed by the coupling at 110 to 120° C. for 8 to 9 hours.

8. The production method of claim 6, wherein step 2) is conducted by performing the condensation reaction of 10 to 200 parts by weight of trichloroacetyl isocyanate on the basis of 100 parts by weight of the silica, in the presence of dibutyltin dilaurate at 80 to 90° C. for 8 to 9 hours.

9. The production method of claim 6, wherein step 3) is performed by adding 1 to 500 parts by weight of the monomer and the crosslinking agent on the basis of 100 parts by weight of the silica, followed by the polymerization reaction for polymer at 65 to 70° C. for 12 to 13 hours.

10. The production method of claim 9, wherein the crosslinking agent has a content of 0.1 to 50 wt % on the basis of the total weight of the monomer and the crosslinking agent.

11. The production method of claim 6, wherein step 3) further includes adding a Mo(CO)6 catalyst.

12. A moisturizing cosmetic composition comprising the silica/zwitterionic polymer composite nanoparticle of claim 1.

13. A hydrogel moisturizing patch comprising the silica/zwitterionic polymer composite nanoparticle of claim 1.
